(19) United States
(12) Patent Application Publication
(10) Pub. No.: US 2010/0190844 A1
(43) Pub. Date: Jul. 29, 2010

(54) PROCESS FOR PREPARATION OF AGLUCONE ISOFLAVONES

(75) Inventors: Kung-Ta Lee, Taipei (TW); Ching-Jang Huang, Taipei (TW); Lun-Cheng Kuo, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN UNIVERSITY, Taipei (TW)

(21) Appl. No.: 12/358,938

(22) Filed: Jan. 23, 2009

(51) Int. Cl. A61K 31/352; C12P 17/06

(52) U.S. Cl. 514/456; 435/125

(57) ABSTRACT

The present invention provides a process for producing a composition containing a high concentration of aglucone isoflavones, which comprises adding glucone isoflavones to the fermentation of microorganisms which are generally recognized as safe, and can express or produce β-glycosidase on a soy-based substrate. The present invention also provides a composition containing a high concentration of aglucone isoflavones produced in accordance with the process of the invention.

### FIELD OF THE INVENTION

The present invention relates to a process for producing a composition having high phytoestrogenic activity. In particular, the invention relates to a process for producing a composition having a high concentration of aglucone isoflavones. The present invention also relates to a composition having a high concentration of aglucone isoflavones.

### BACKGROUND OF THE INVENTION

Soy isoflavones, e.g., genistein and daidzein, are structurally similar to estrogen and are thus called “phytoestrogens.” They have been implicated in some health-enhancing processes, such as prevention of cancers (Cappelletti et al., 2000: Miura et al., 2002; Ravindranath et al., 2004), lowering of risk of cardiovascular diseases (Anthony et al., 1996; Goodman-Gruen and Kritz-Silverstein 2001), improvement of bone health (Cotter and Cashman 2003; Weaver and Cheong 2005), and alleviation of post-menopausal syndromes. Particularly, isoflavones have been clinically confirmed to have therapeutic effects on osteoporosis, including inhibition of bone loss and bone resorption and promotion of bone formation in post-menopausal women, with no adverse effects observed on the breast and uterus.

The contents of isoflavones in plants are very low. For instance, soy contains merely about 0.1% (w/w) of isoflavones. In addition, the isoflavones in plants are in the form of glucone isoflavones, which should be hydrolyzed into aglucone isoflavones so that it can be absorbed by the intestine (Setchell et al., The Journal of Nutrition 2001, Vol. 131, 1362S-1375S.) Research has shown that the concentration of isoflavones in blood after the administration of aglucone isoflavones is 2 to 5 times higher than that after the administration of glucone isoflavones (Izumi et al., The Journal of Nutrition 2000, Vol. 130, 1695-1699).

Nonetheless, the body's capability of hydrolyzing isoflavone glucone in the intestine decreases with age, which causes health problems in senior population. One solution to such problems is to directly provide aglucone isoflavones in food or food supplements.

U.S. Pat. Nos. 5,320,949, 5,352,384, 5,637,561, 5,637,562, 5,726,034 and 5,821,361 disclose processes for converting materials containing glucone isoflavones to materials containing aglucone isoflavones with β-glucosidase derived from plants or microorganisms. Other patent documents disclose processes for hydrolyzing glucone isoflavones into aglucone isoflavones with β-glucosidase derived from microorganisms. For instance, U.S. Pat. No. 5,554,519 teaches a process for fermentating soybeans with Saccharopolyspora erythraea to produce genistein; PCT Patent Application No. PCT/JP94/02103 teaches a process for producing aglucone isoflavones from pulse crops fermentated with koji; and Japanese Patent Application 2003070439 teaches the addition of purified β-glucosidase produced by Penicillium multicolor and other koji to the process of natto production to enhance deglycosylation of isoflavones.

It has also been disclosed that the fermentation of black soybeans with Bacillus subtilis natto converts glucone isoflavones in the black soybeans into aglucone isoflavones (Kuo et al., 2006).

However, the above processes only produce products having low contents of aglucone isoflavones, and cannot be used for mass production for applications in the medicinal field or the field of health foods. Thus, there remains a need for a process which produces high concentrations of aglucone isoflavones.

### SUMMARY OF THE INVENTION

The invention provides a process for producing a composition comprising a high concentration of aglucone isoflavones, which comprises adding glucone isoflavones to the fermentation of a soy-based substrate with microorganisms which are generally recognized as safe, and can express or produce β-glycosidase.

The invention further provides a process for producing aglucone isoflavones wherein the glucone isoflavones in a soy-based material are deglycosylated with β-glucosidase, with the improvement that glucone isoflavones are added.

The invention also provides a composition comprising a high concentration of aglucone isoflavones produced in accordance with the processes of the invention.

### BRIEF DESCRIPTION OF THE DRAWING

FIG. 1 Deglycosylation of isoflavone glycosides in flask.

FIG. 2 Deglycosylation of isoflavone glycosides in flask.

FIG. 3 Production of aglucone isoflavones in flask by fed-batch.

FIG. 4A Cell numbers in continuous production of aglucone isoflavones in bioreactor.

FIG. 4B Isoflavone concentrations in continuous production of aglucone isoflavones in bioreactor.

FIG. 5A Phytoestrogenic activity (α-receptor) during bioconversion.

FIG. 5B Phytoestrogenic activity (β-receptor) during bioconversion.

### DETAILED DESCRIPTION OF THE INVENTION

The present invention provides a process for preparing a composition containing a high concentration of aglucone isoflavones, which comprises adding glucone isoflavones to the fermentation of a soy-based substrate with microorganisms which are generally recognized as safe, and can express or produce β-glycosidase.

As used herein, the term “glucone isoflavones” refers to plant glucone isoflavones, e.g., daidzin and genistin, and “aglucone isoflavones” refers to plant aglucone isoflavones, e.g., daidzein and genistein.

As used herein, the term “soy-based substrate” refers to a substrate of any form that contains or is derived from soybeans. The soy-based substrate suitable for use in accordance with the invention includes, but is not limited to, pulse crops, soybean meal, soymilk, soybean flour, black soybean meal, black soymilk, black soybean flour, and the like.

The fermentation of the soy-based substrate with microorganisms which are generally recognized as safe, and can express or produce β-glycosidase can be performed in accordance with the processes known in the art, e.g., those taught in the references mentioned supra. The soy-based substrate can be sterilized in accordance with conventional processes, e.g., by autoclaving, before the inoculation of mircoorganisms.

The microorganisms are inoculated so that their concentration range from 10² to 10⁷ cfu/ml at the beginning of fermentation, and range from 10⁵ to 10¹² cfu/ml at the end of the fermentation. Preferably, the concentration of the microorganisms in the fermentation ranges from 10³ to 10⁶ cfu/ml at the beginning of fermentation, and range from 10⁸ to 10¹⁰ at the end of the fermentation. Most preferably, the concentration of the microorganisms in the fermentation is 10⁵ cfu/ml at the beginning of fermentation, and is 10⁹ at the end of the fermentation.

Microorganisms which are generally recognized as safe, and can express or produce β-glycosidase can be used in the fermentation according to the invention. Such microorganisms include, but are not limited to, Actinomucor elegans, A. taiwanensis, Aspergillus awamori, A. oryzae, A. sojae, Bacillus subtilis, B. subtilis (natto), Bifidobacterium animalis, B. breve, B. infantis, B. longum, B. thermophilum, Candida spp, Debaryomyces spp, Ganoderma lucidum, Lactobacillus acidophilus, L. casei, L. delbrueckii, L. paracase, L. plantarum, Lactococcus lactis, Monascus spp, Mucor spp, Rhizopus azygosporus, Saccharomyces spp, Saccharopolyspora erythraea, Streptococcus thermophilus and Zygosaccharomyces spp. The strain Bacillus subtilis natto is the most preferably.

After inoculation of the bacteria, the cultivation is performed under suitable conditions for a period. For instance, the cultivation can be performed at a temperature from about 10° C. to about 60° C. The cultivation can also be performed at a shaking rate of about 20 rpm to about 2000 rpm. Preferably, the cultivation is performed at the temperature of about 20° C. to about 40° C., and at a shaking rate of about 50 rpm to about 1000 rpm. Most preferably, the cultivation is performed at the temperature of about 35° C. to about 40° C., and at a shaking rate of about 120 rpm to about 200 rpm.

The fermentation is maintained for a period sufficient for converting glucone isoflavones into aglucone isoflavones. The time needed will vary with the cultivation temperature, the shaking rate, the concentrations of the bacteria, etc., and can be determined by persons of ordinary skill in the art. In general, the time may range from about 8 hours to 240 hours, preferably from about 20 hours to 120 hours.

β-glucosidase is conventionally used for the production of aglucone isoflavones by deglucosylating glucone isoflavones. However, the enzyme is inhibited in the reaction by the resultant glucose, which makes it difficult to obtain a high concentration of aglucone isoflavones.

It was surprisingly found that a high concentration of aglucone isoflavones are produced during the fermentation of a soy-based substrate with the microorganisms in accordance with the invention if glucone isoflavones are added during the fermentation. It is believed that glucose produced as a result of the deglucosylation of the glucone isoflavones added to the fermentation of a soy-based substrate with the microorganisms in accordance with the invention serves as a carbon source for and thus enhance the proliferation of the bacteria, which in turn enables the production of a high concentration of aglucone isoflavones. Compared with conventional processes where materials containing glucone isoflavones are fermented with microorganisms which produce β-glucosidase needed for deglucosylation, the process of the present invention significantly improves the amount of the aglucone isoflavones produced.

The invention further provides a process for producing aglucone isoflavones wherein the glucone isoflavones in a soy-based material are deglycosylated with β-glucosidase, with the improvement that glucone isoflavones are added.

The glucone isoflavones can be added to the fermentation of the soy-based substrate with the microorganisms in accordance with the invention by batch, fed-batch or continuous feeding.

As is well known in the art, the fermentation may be carried out in any suitable containers, which include but are not limited to, a flask, a fermentor and a bioreactor.

The processes of the invention result in products of high concentrations of aglucone isoflavones, which exhibit high phytoestrogenic activity, i.e., high β-receptor affinity and low α-receptor affinity, and are thus suitable for applications in the medicinal field or the field of health food.

The invention further provides a composition comprising a high concentration of aglucone isoflavones, which is produced in accordance with the processes of the invention.

Because of its high phytoestrogenic activities, the composition of the invention can be used in medicines or health foods to maintain bone density, lower the risk of cardiovascular diseases, prevent the occurrence of cancers (such as breast cancer and prostate cancer), and alleviate post-menopausal syndrome.

The product of the processes of the invention can be subjected to conventional processing to be in the forms suitable for use in medicines or health foods. In accordance with different needs, the composition of the invention can be formulated in the form of tablet, pellet, powder, solution, syrup, or the like. For instance, the composition can be lyophilized in a conventional manner.

The specific examples below are to be construed as merely illustrative and do not limiting the remainder of the disclosure. It is believed that persons having ordinary skill in the art can, based on the basis of the descriptions herein, utilize the present invention to its fullest extent.

### EXAMPLES

### Example 1

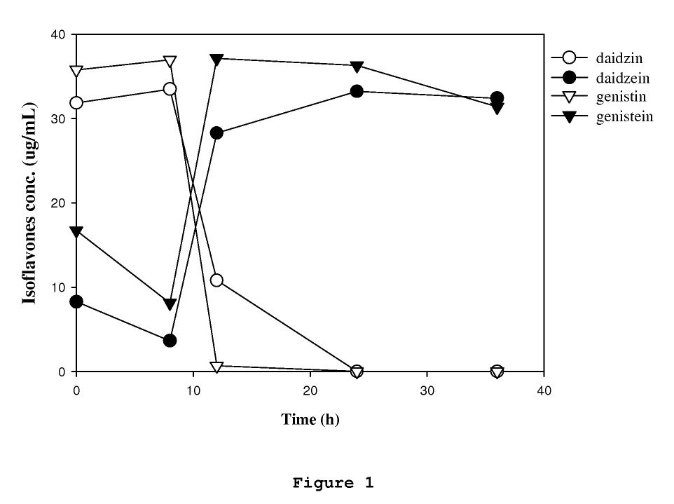

In a 500 ml flask, 100 ml culture medium containing 5% (w/v) of pulse crops was prepared, and subjected to autoclaving at 121° C. for 15 minutes. Bacillus subtilis natto was then inoculated into the medium and cultivated in a shaker at 37° C., 125 rpm. At the beginning of the cultivation, the viable cell number was 9.6×10³ cfu/ml. After 12 h, number was of 6×10⁸ cfu/ml, which was maintained to the end of the cultivation (24 h after inoculation). p-nitrophenyl-β-D-glucoside (PNPG) was used as substrates to determine the enzyme activity of β-glucosidase throughout the cultivation process. The β-glucosidase activity was detectable 8 h after inoculation and increased to the maximum (3.3 U/ml) at 15 to 18 h, then the β-glucosidase activity decreased to 0.3 U/ml after 21 h of cultivation. The concentration of daidzin and genistin were 76.5 and 82.7 μM, respectively, at the beginning of the cultivation, and started to decrease after 8 h of cultivation. At 12 h, the concentrations of daidzin and genistin were 25.9 and 1.5 μM, respectively, while those of daidzein and genistein increased from 28.5 to 111.6 μM and 54.2 to 101.6 μM within 12 h, respectively (FIG. 1).

### Example 2

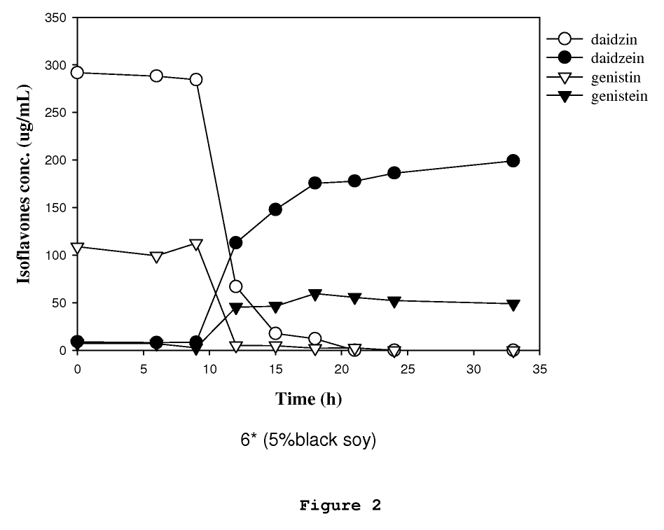

100 ml culture medium of 5% (w/v) pulse crops was prepared in a 500 ml flask and materials containing glucone isoflavones (10 mg of genistin and 30 mg of daidzin were contained) were added. After being autoclaved at 121° C. for 15 minutes, Bacillus subtilis natto was then inoculated into the medium and cultivated in a shaker at 37° C., 125 rpm. At the beginning of the cultivation, the viable cell number is 1.0×10⁵ cfu/ml. After 12 h of cultivation, the number was 6.9×10⁸ cfu/ml, which was maintained to the end of the cultivation (24 h after inoculation). The enzyme activity of β-glucosidase was monitored throughout the cultivation process. The β-glucosidase activity was detectable 8 h after inoculation and increased gradually. It achieved the maximum 12 h after inoculation and decreased to 1.7 U/ml after 15 h of cultivation. Between 18 and 21 h the enzyme activity remained at 1.0 U/ml. Deglycosylation of isoflavone glycosides by Bacillus subtilis natto throughout the cultivation was investigated. The concentration of daidzin and genistin were 700.3 and 251.7 μM, respectively, at the beginning of the cultivation. After 8 h of cultivation, daidzin and genistin started to deglycosylate to daidzein and genistein, respectively. 12 h after inoculation, the concentrations of daidzin and genistin decreased to 160.2 and 11.6 μM, respectively, while those of daidzein and genistein increased from 35.5 to 444.4 μM and 26.9 to 168.0 μM within 12 h, respectively. 21 h after inoculation, all daidzin and genistin were converted to daidzein and genistein with a concentration of 699.6 , and 206.3 μM, respectively (FIG. 2).

### Example 3

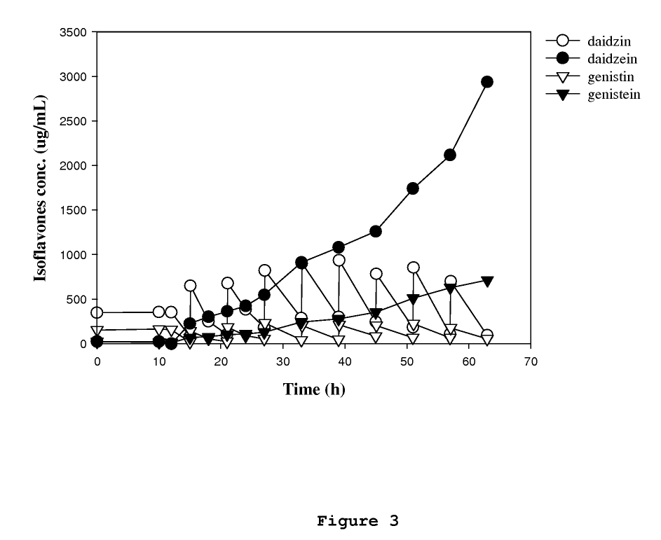

100 ml culture medium of 5% (w/v) pulse crops was prepared in a 500 ml flask and materials containing heat-extracted glucone isoflavones (containing 10 mg of genistin and 30 mg of daidzin) were added. After being autoclaved at 121° C. for 15 minutes, 1 ml of pre-cultivated Bacillus subtilis natto was inoculated into the medium and cultivated in a shaker at 37° C., 250 rpm. After 15 h of cultivation, glucone isoflavones were fed every 6 h for a total of 8 feeds and a total of 266 mg of genistin and 1036 mg of daidzin were added. The results showed that the bacteria proliferated between 1 and 10 h, where 8.5×10⁴ cfu/ml viable cells at the beginning of the cultivation increased to 1.7×10⁹ cfu/ml at 10 h, and entered a stationary phase of 1.4×10⁸ to 2.7×10⁸ cfu/ml until the end of the cultivation (63 h after inoculation). The β-glucosidase activity was detectable 12 h after inoculation and increased gradually. It achieved the maximum of 9.7 U/ml 27 h after inoculation and then decreased to 6.3 U/ml after 45 h of cultivation. After 8 feeds, i.e., 63 h after inoculation, the concentration of daidzein and genistein in the medium reached 11548.0 and 2636.1 μM, respectively (FIG. 3). The variation of pH was from 7.5 to 8.0. After lyophilization of the products, each gram of powder contained 66 mg of daidzein and 24 mg of genistein, which was 100 times the content in soybeans.

### Example 4

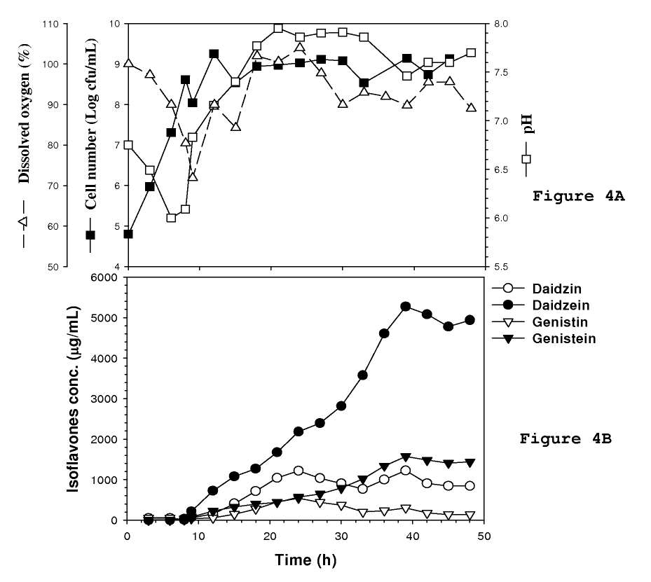

2 L culture medium of 5% (w/v) pulse crops was prepared in a 5 L fermentor and subjected to autoclaving at 121° C. for 15 minutes. 10 ml of Bacillus subtilis natto pre-cultivated in a flask was inoculated into the medium to cultivate under aeration and agitation at 37° C., 700 rpm. The results showed that the bacteria proliferated between 1 and 12 h, where 7.2×10⁵ cfu/ml viable cells at the beginning of the culture increased to 2.3×10⁹ cfu/ml at 12 h, and entered a stationary phase until the end of the cultivation (48 h after inoculation) (FIG. 4A). Continuous substrate feeding of glucone isoflavones was conducted after 8 h of inoculation. Glucone isoflavone solution was fed at a rate of 1.5 ml per minute for 12 hours from 8 h to 20 h after inoculation. Daidzein and genistein were 2464 and 541 μM, respectively at the end of feeding (FIG. 4B).

### Example 5

Samples of fermented medium undergoing aglucone isoflavone conversion in bioreactor by fed-batch were taken from Example 3 at 0, 21, 51 and 63 h after inoculation and lyophilized and put to estrogenic activity tests. The concentrations of the estrogen activity determining samples are 10 ng/ml and 100 ng/ml in 50% ethanol solution. CHO-K1 cells (ATCC, CCL 61), cultivated in 96-well plates at a density of 2.5×10⁴ cells/well with Ham's F-12 medium containing 10% fetal calf serum, were incubated at 37° C., 5% CO₂ for 24 hours. The cells were then transfected with reporter vectors pBK-CMV-Gal4-hERα (or β) and pBK-CMV-(UAS)4-tk-alkaline phosphatase (AP) for 5 hours. After that, the medium containing transfection agents was removed. The samples to be tested were diluted with the culture medium. 17□-estradiol (E2, Sigma, E2758) was used as control. Both the estrogenic activity determining samples and the control were subjected to estrogen activation reaction for 48 hours. A quantity of the medium was taken from the culture to determine alkaline phosphatase (AP) activity. 4-Nitrophenyl phosphate disodium salt hexahydrate ( pNPP ) was used as the substrate to react with the samples for 15 minutes in the 96-well plate and the absorption values were then determined at 405 nm.

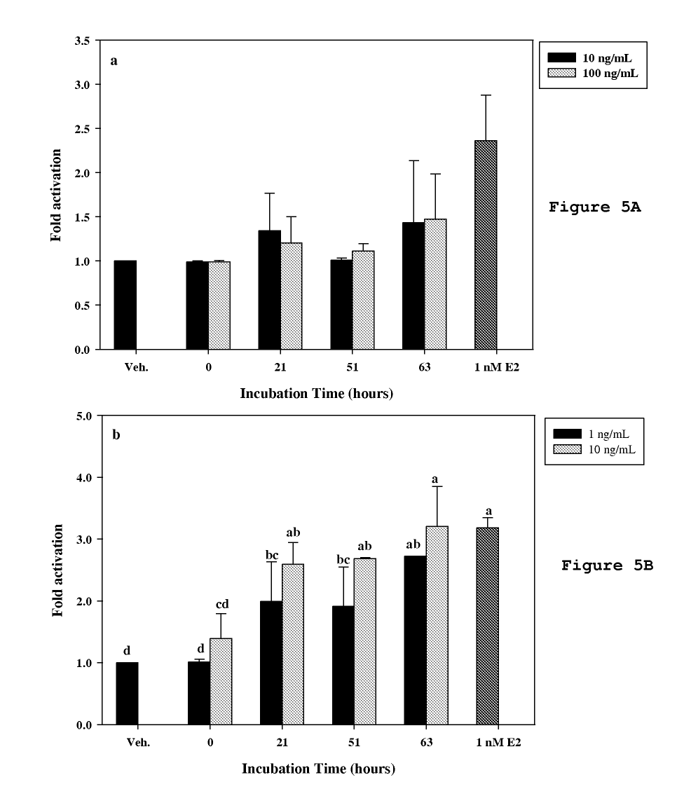

The data reveal that, compared to 17□-estradiol, which is clinically used estrogen, the products obtained by the aglucone isoflavone conversion of the present invention do not increase the expression of α-receptor, whether at 10 ng/ml or 100 ng/ml (FIG. 5A). This means that the converted products will not increase the risk of occurrence of breast cancer related diseases. Meanwhile, the samples increase the expression of □-receptor as the conversion time increases, whether at 10 ng/ml or 100 ng/ml (FIG. 5B). The results suggest that the products have a positive effect on increasing bone calcium absorption and inhibiting the occurrence of cardiovascular diseases.

What is claimed is:

1. A process for producing a composition comprising a high concentration of aglucone isoflavones, which comprises adding glucone isoflavones to the fermentation of a soy-based substrate with microorganisms which are generally recognized as safe, and can express or produce β-glycosidase.

2. The process according to claim 1, wherein the glucone isoflavones are added by batch, by fed-batch or by continuous substrate feeding.

3. The process according to claim 1, wherein the microorganisms are selected from the group consisting of Actinomucor elegans, A. taiwanensis, Aspergillus awamori, A. oryzae, A. sojae, Bacillus subtilis, B. subtilis (natto), Bifidobacterium animalis, B. breve, B. infantis, B. longum, B. thermophilum, Candida spp, Debaryomyces spp, Ganoderma lucidum, Lactobacillus acidophilus, L. casei, L. delbrueckii, L. paracase, L. plantarum, Lactococcus lactis, Monascus spp, Mucor spp, Rhizopus azygosporus, Saccharomyces spp, Saccharopolyspora erythraea, Streptococcus thermophilus, and Zygosaccharomyces spp.

4. The process according to claim 3 wherein the microorganism is Bacillus subtilis natto.

5. The process according to claim 1, wherein the fermentation is performed at a temperature of about 10° C. to about 60° C., at a shaking rate of about 20 rpm to about 2000 rpm for about 8 hours to about 240 hours.

6. The process according to claim 5, wherein the fermentation is performed at a temperature of about 20° C. to about 4° C., at a shaking rate of about 50 rpm to about 1000 rpm for about 20 hours to about 240 hours.

7. The process according to claim 6, wherein the fermentation is performed at a temperature of from about 35° C. to about 40° C., at a shaking rate of about 120 rpm to about 200 rpm for about 20 hours to about 120 hours.

8. A process for producing aglucone isoflavones which comprises deglycosylating the glucone isoflavones in a soy-based material with β-glucosidase, with the improvement that glucone isoflavones are added.

9. The process according to claim 8, wherein the glucone isoflavones are added by batch, by fed-batch or by continuous substrate feeding.

10. A composition comprising a high concentration of aglucone isoflavones, which is produced in accordance with the process of claim 1.

11. The composition of claim 10, wherein the aglucone isoflavones are daidzein and/or genistein.

12. A composition comprising a high concentration of aglucone isoflavones, which is produced in accordance with the process of claim 8.

13. The composition of claim 12, wherein the aglucone isoflavones are daidzein and/or genistein.
